(19) United States
(12) Patent Application Publication
(10) Pub. No.: US 2009/0304735 A1
(43) Pub. Date: Dec. 10, 2009

(54) Immunogenic Compositions

(76) Inventors: Gabrielle Therese Belz, Victoria (AU); William Ross Heath, Victoria (AU)

Correspondence Address: KLAUBER & JACKSON, 411 HACKENSACK AVENUE, HACKENSACK, NJ, 07601, US

(21) Appl. No.: 12/227,515

(22) PCT Filed: May 18, 2007
(86) PCT No.: PCT/AU2007/000691

§ 371 Date: Jul. 13, 2009

Related U.S. Application Data
(60) Provisional application No. 60/801,998, filed on May 19, 2006.

(51) Int. Cl. A61K 39/145; A61K 39/12; A61K 39/245; A61K 39/02; A61K 39/285; A61P 35/00; A61P 37/00

(52) U.S. Cl. 424/199.1; 424/206.1; 424/236.1

(57) ABSTRACT

The present invention relates to an immunogenic composition for raising an immune response to an antigen, the composition comprising the antigen and a targeting moiety specific for lymph-resident dendritic cells. Use of the immunogenic composition in a vaccine and methods of boosting an immune response using the composition are also provided. Conversely, the invention also relates to immunogenic composition for raising an immune response to an antigen, the composition comprising the antigen and a targeting moiety specific for tissue-derived dendritic cells and a vaccine comprising said composition.

### CROSS REFERENCE TO RELATED APPLICATIONS

The present application is a National Stage Application claiming the priority of co-pending PCT Application No. PCT/AU2007/000691 filed May 18, 2007, which in turn, claims priority from U.S. Provisional application Ser. No. 60/801,998 filed May 19, 2006. Applicants claim the benefits of 35 U.S.C. § 120 as to the PCT application and priority under 35 U.S.C. § 119 as to the said U.S. Provisional application, and the entire disclosures of both applications are incorporated herein by reference in their entireties.

### FIELD

The present invention relates to immunogenic compositions and vaccines and more particularly, though not exclusively to immunogenic compositions, vaccines and kits for use in prime-boost immunisation strategies. The invention also relates to methods for generating an immune response using such immunogenic compositions, vaccines or kits.

### BACKGROUND

Vaccines traditionally consisted of live attenuated pathogens, whole inactivated organisms or inactivated toxins. In many cases these vaccines have been successful at inducing immune protection based on antibody mediated responses. However, many infections and malignant diseases, e.g., HIV, HCV, TB, cancer and malaria, require the induction of cell-mediated immunity (CMI). Despite the development of new approaches to vaccine development, such as recombinant protein subunits, synthetic peptides, protein polysaccharide conjugates, DNA vaccines and the use of recombinant viral vectors that mimic the antigenicity of infectious agents, a general problem is that vaccines are often poorly immunogenic. Therefore, there is a continuing need for the development of ways to enhance the immunogenicity of vaccines.

Prime-boost vaccination is often used to enhance the immunogenicity of a vaccine, i.e. an individual is vaccinated more than once, to elicit a secondary immune response. In prime-boost vaccination the “prime” stage, i.e. the first vaccination step, involves presentation of antigen to naïve immune cells and the generation of memory B and T cells. Without subsequent presentation of antigen these memory cells reduce in number. Furthermore, a single immunization often induces such a small response that additional immunizations are required. Accordingly to improve the ability to produce an immune response supplementary “boost” vaccination is required, to generate more memory B and T cells which provide for enhanced and accelerated secondary responses should the vaccinated individual undergo subsequent exposure to the antigen.

Prime boost strategies have been used for many years, for example to vaccinate against measles and mumps. More recently, heterologous prime-boost vaccination, where a different antigen is used in the booster, has been shown to be more efficient in inducing a CMI response than use of a single vector.

It is an aim of a preferred embodiment of the present invention to provide an immunogenic composition for use in a booster vaccine to provide increased potency over vaccines disclosed in the art, whether referred to herein or otherwise and to provide a method of enhancing the efficacy of prime boost vaccination.

### SUMMARY

In a first aspect the invention provides an immunogenic composition for raising an immune response to an antigen, the composition comprising the antigen and a targeting moiety specific for lymph-resident dendritic cells.

The inventors have determined that whilst both naïve and memory killer (CD8+) T cells respond to viral antigens presented by lymph-resident dendritic cells (DCs) surprisingly only naïve cells respond efficiently to tissue-derived DC. Memory killer T cells respond efficiently to antigens presented by lymph-resident DCs, but are poorly responsive to antigens presented by tissue-derived DCs. Accordingly, the inventors propose that targeting an antigen to lymph-resident DCs will increase the efficiency of a booster vaccination. This is particularly surprising because memory T cells were always considered to be more responsive and sensitive to stimulation than naïve T cells.

In a second aspect the invention provides a booster vaccine comprising an immunogenic composition according to the first aspect of the invention.

In a third aspect the invention provide a kit comprising a first vaccine and a booster vaccine comprising the immunogenic composition according to the first aspect of the invention.

In a fourth aspect the invention provides a method of inducing an immune response in an individual comprising administering to the individual a booster vaccination comprising an immunogenic composition according to the first aspect of the invention.

In a fifth aspect the invention provides use of an immunogenic composition according to a first aspect of the invention in the manufacture of a medicament for administering to an individual to induce an immune response.

In a sixth aspect the invention provides an immunogenic composition for raising an immune response to an antigen, the composition comprising the antigen and a targeting moiety specific for tissue-derived dendritic cells.

The inventors' recognition that only naïve cells respond efficiently to tissue-derived DC allows them to propose that targeting an antigen to tissue-derived DCs could provide enhanced protection from pathogens. If a pathogen normally induces a specificity that is not good at recognising and fighting the pathogen, then by targeting the right sort of antigen to tissue-derived DCs you could promote expansion of a new specificity without competition by the old (non-effective) specificity.

In a seventh aspect the invention provides method of inducing an immune response in an individual comprising administering to the individual a primary or booster vaccination comprising an immunogenic composition according to the sixth aspect of the invention.

In an eight aspect the invention provides use of an immunogenic composition according to a sixth aspect of the invention in the manufacture of a medicament for administering to an individual to induce an immune response.

### BRIEF DESCRIPTION OF THE FIGURES

FIG. 1a shows the phenotype of naïve and memory CD8⁺ T cells analysed for expression of activation markers CD25, CD69, CD44 and CD62L.

FIG. 1b shows the results of T cell proliferation assays for DC subsets cultured with naïve or memory gBT-I CD8+ CSFE-labelled transgenic T cells specific for HSV glycoprotein B (gB). DC subsets were derived from mice infected with WSN-gB 3 days previously.

FIG. 1c shows the results of T cell proliferation assays for DC subsets cultured with CFSE-labelled endogenous memory CD8⁺ T cells. Memory T cells were derived from mice infected with WSN-gB 3 (upper) or HKx31/PR8 (lower) 6 months previously.

FIG. 2a shows the results of T cell proliferation assays for DC cultured with naïve gBT-I CD8⁺ CSFE-labelled transgenic T cells specific for HSV glycoprotein B (gB). DC subsets were derived from mice infected with WSN-gB at various times.

FIG. 2b is a schematic representation of the extent of antigen presentation to T cells by purified DC subsets as mapped by direct ex vivo analysis. It also outlines the protocols used in FIGS. 2c and 2d.

FIG. 2c shows flow cytometric analysis of division of CFSE labelled naïve or memory gBT-I CD8⁺ T cells transferred into uninfected mice (middle panel) or those infected with WSN-gB ten (upper panel) or three (lower panel) days previously.

FIG. 3a and FIG. 3b show the responsiveness of mixed cultures of naïve and memory T cells to lung DC (FIG. 3a) and lymph-resident DC (FIG. 3b).

FIG. 3c shows the responsiveness of naïve and memory T cells to mixtures of different DC subtypes from the medistinal lymph node (MLN) 3 days after viral infection.

FIG. 3d shows the responsiveness of naïve and memory T cells to SSIEFARL peptide coated CD8 DC and CD11b⁻DC from MLN.

FIG. 3e shows the responsiveness of naïve and memory T cells to SSIEFARL peptide coated CD8⁺ and CD8⁻DEC205⁺ (Langerhans cells and dermal) DC from skin draining LN.

FIG. 3f shows the responsiveness of naïve and memory T cells to SSIEFARL peptide coated CD8⁺ and CD8⁻CD11b⁻ DC from lymph node resident and lung-derived DCs from influenza infected mice.

Each figure shows proliferated gBT-I CD8⁺ T cells counted by flow cytometry. Data are one representative of two experiments for each data set.

FIG. 4 shows flow cytometric profiles of CD8+ DCs (black line) and lung-derived DCs (CD8⁻CD11b⁻, grey) enriched from the mediastinal LN of mice infected with influenza HKx31 virus three days previously. Cells were stained with antibodies against CD11c, CD11b and CD8, together with antibodies against B7-H1, B7-H2, B7-DC, B7-RP, B7-1, B7-2 or BTLA-4.

FIG. 5 shows purified CD8α DCs or CD11b⁻CD8⁻ lung-derived DCs from the mediastinal lymph nodes of WSN-gB infected mice cultured with CFSE-labelled naïve or memory gBT-I in the presence or absence of 1 mg/ml of a blocking monoclonal antibody to CD70 (clone FR70). After 60 h, proliferation was assessed by flow cytometry. Data is expressed as reduction in proliferation relative to the isotype control and is pooled from 3 independent experiments. Note that memory T cells do not respond to lung-derived DCs so this value was not determined (n.d.).

FIG. 6 shows the number of memory or naïve T cells that have proliferated in competition with either memory or naïve T cells in mice infected intranasally with WSN-gB and their tissues analysed 10 days later (FIGS. 6 a to c), or infected intravenously and analysed 8 days later (FIG. 6 d). Above each graph is listed the competing population versus the responding population. Numbers on the y-axis indicate the number of the responding population detected at the end of the experiment. Numbers on the x-axis indicate the number of the competing cells added to the mice. The data presented show results of individual experiments with at least two mice per experimental point. In FIG. 6e, mice were left untreated (left panel, none) or adoptively transferred with 2.2×10⁶ CD44^{high}CD62^{high} memory CD8⁺ T cells purified from mice that had been infected with influenza HKx31 at least 12 weeks previously (left panel, Memory cells; right panel). Twenty-four hours later, mice were infected with HKx31 intranasally. After 10 days, spleens were analysed for the number of the endogenous (left panel) or transferred memory (right panel) CD8⁺ T cells specific for D^{b}NP₃₆₆₋₃₇₄ or D^{b}PA₂₂₄₋₂₃₃. Data are pooled from 4 experiments, with each circle representing an individual mouse. In FIG. 6f, mice were left untreated (left panel, None) or adoptively transferred with 2.2×10⁶ CD44^{high}CD62^{high} memory CD8⁺ T cells purified from mice that had been infected with influenza HKx31 at least 12 weeks previously (Left panel, Memory cells; middle panel). Twenty-four hours later, mice were infected with HKx31 intravenously. After 8 days, spleens were analysed for the number of the endogenous (left panel; right panel) or transferred memory (middle panel) CD8⁺ T cells specific for D^{b}NP₃₆₆₋₃₇₄ or D^{b}PA₂₂₄₋₂₃₃. Values for naïve uninfected mice (right panel). Data are pooled from 2 experiments, with each circle representing an individual mouse.

FIG. 7 shows flow cytometry analysis of purified DC co-cultured with gBT-I CD8⁺ CFSE-labelled naïve (upper row) or various types of memory transgenic (middle two rows) or endogenous (lower row) CD8⁺ T cells specific for gB. The histograms are representative of 2 experiments with similar results and show proliferation of the T cell population. The percent and number (parenthesis) of proliferating cells for each plot are indicated.

### DETAILED DESCRIPTION

DC involvement in T cell responses starts with the capture of antigen in peripheral tissues followed by migration to draining lymph organs and presentation of antigen for T cell priming. DCs are the most potent antigen presenting cells (APCs) used by the immune system.

DCs are a heterogeneous cell type consisting of multiple subsets. Some reside permanently within lymphoid organs (lymph-resident), while others (tissue-derived) are found in non-lymphoid tissues and only traffic to local lymph nodes upon antigen capture.

DCs are potent APCs for several immune responses. Different DC subsets and DCs at different stages of development or activation express distinct surface molecules and secrete cytokines that selectively determine the type of immune response which is induced. For example, after lung infection with influenza virus two types of dendritic cells are responsible for activating naïve virus-specific killer T cells [Belz, GT. et al., PNAS, vol. 101, no. 23 p 8670-8675]. These dendritic cells are identified as CD205⁺CD11b⁻CD8alpha⁻ (lung-derived) and CD205⁺CD11b⁻CD8alpha⁺ (lymph-resident) dendritic cells.

Because memory T cells have been reported to have less co-stimulatory requirements than naïve T cells the inventors investigated whether memory T cells might respond to additional DC subsets to the two types recognised by naïve T cells during lung infection with influenza virus.

Unexpectedly memory T cells were found to be less broadly responsive than naïve T cells. Memory T cells failed to proliferate in response to antigen presentation by lung derived DC (CD8⁻) but did respond to antigen presentation by lymph-resident DC (CD8⁺). This was the case whether the memory T cells were produced in vitro or in vivo by exposure to virus infection.

Further experiments to quantitatively assess the difference in stimulatory capacity of lung-derived DC for naïve and memory T cells revealed an approximately 10-fold reduction in the sensitivity of memory T cells to lung-derived DC. Thus while lung-derived DC could not stimulate memory T cells to influenza virus during infection, this was not due to a complete failure of the population to activate memory, but rather a 10-fold reduced capacity to stimulate. Realistically, however, this difference could mean that most natural stimuli are ineffective at stimulating memory T cells when presented on lung-derived DC.

The inventors considered whether these findings extend beyond the lung-derived DC. They compared antigen presentation by skin derived DC and found that again, while lymph-resident DC stimulated both naïve and memory cells equivalently, skin-derived DC were 10-fold less efficient at stimulating memory T cells.

The inventors also found that trafficking (tissue-derived) DCs are critical for naïve T cell stimulation when competing memory cells are present. This explains why naïve T cell responses could be detected despite the presence of preformed memory for lung infection with influenza virus.

Accordingly the inventors propose that booster vaccine should target the antigen to lymph-resident DCs so as to stimulate memory T cells. Any antigen directed to non-lymph-resident DCs is effectively wasted in a booster vaccine, since the ability of memory T cells to respond to trafficking DCs is compromised, antigen processed by tissue DCs will not be capable of stimulating memory T cells. The present invention provides for increased efficiency in booster vaccinations.

The studies also show naïve T cells to be more sensitive than memory T cells for stimulation by tissue-derived DC and are equivalent to naïve T cells in their response to lymph-resident DCs. This questions the long-held paradigm that memory T cells have fewer co-stimulatory requirements than naïve T cells.

The inventors propose that the converse of the invention may also hold true, that is that specifically excluding lymph-resident DCs from attach by antigen and targeting antigen to tissue-derived DCs may be effective in raising a naive immune response although a primary immune response has previously been raised to an antigen and memory cells exist. This may be particularly convenient if an antigen (e.g. a pathogen) that normally induces a specificity that is not good at recognising and fighting the pathogen.

The present invention relates to an immunogenic composition. As referred to herein an immunogenic composition is any composition or formulation that is capable of generating an immune response.

An immune response is the body's reaction to foreign antigens. This response may neutralize or eliminate the antigens and provide protective immunity against future encounters with microbes or toxins.

By “immune response” or “immunity” as the terms are interchangeably used herein, is meant the induction of a humoral (i.e., B cell) and/or cellular (i.e., T cell) response. Suitably, a humoral immune response may be assessed by measuring the antigen-specific antibodies present in serum of immunized animals in response to introduction of the antigen into the host. The immune response may be assessed by the enzyme linked immunosorbant assay of sera of immunized mammals, or by microneutralization assay of immunized animal sera. A CTL assay can be employed to measure the T cell response from lymphocytes isolated from the spleen or other organs of immunized animals.

The immunogenic composition of the present invention provides a killer T cell or CTL response and may optionally provide a T helper cell response. It may further provide a humoral response.

Persons skilled in the art will appreciate that a humoral response (an antibody response) is the production of immune protection by the generation of B cells, which secrete antibodies in response to antigen (as distinct to the direct action of immune cells or the cellular immune response). Antibodies are molecules produced by a B cell in response to an antigen. When antibodies attach to an antigen they help to destroy the pathogen bearing the antigen (an neutralising response).

Persons skilled in the art will appreciate that a cell mediated immune response is immune protection provided by the direct action of immune cells. A cell mediated response involves T cells, i.e. white blood cells (also known as T lymphocytes) that direct or participate in immune defences. T cells include cytotoxic T cells (also called killer T cells or TK cells), which destroy cells of the body that are infected with foreign antigens. Another T cell subset is the T helper cell or TH cell subset. These function as messengers. They are important for turning on antibody production, activating cytotoxic T cells and for initiating many other immune functions.

A primary immune response as referred to herein is an adaptive response generated on first exposure of an individual to a foreign antigen. Primary responses are characterised by relatively slow kinetics and small magnitude when compared with the responses after a second or subsequent exposure.

A secondary immune response as referred to herein is an adaptive response that occurs upon second exposure of an individual to a foreign antigen. A secondary response is usually characterised by more rapid kinetics and greater magnitude when compared with the primary response.

For this reason the immune system is primed by vaccination. Vaccination is the administration of an antigen preparation in the form of a vaccine to induce protective immunity against infection by microbes bearing that antigen.

Priming is the administration of the initial course of a vaccine intended to induce an immune response and immune memory; it may be followed by a later vaccine dose(s) called a booster.

Priming may also occur upon exposure of the immune system to an infective agent such as a virus.

A booster is a second or subsequent vaccine dose given after the primary dose, to increase immune responses. A booster vaccine may be the same as the primary one, or different (heterologous prime-boost).

Prime-boost is a vaccine regimen in which a primary vaccine injection(s) is followed by booster injection(s) at a later time with the same or a different (heterologous prime-boost) vaccine preparation. A prime-boost combination may induce stronger or different types of immune responses from those seen with the primary immunization.

Naïve cells are mature B or T lymphocytes that have not previously encountered antigen, nor are progeny of antigen stimulated mature lymphocytes. When naïve lymphocytes are stimulated by antigen, they differentiate into effector lymphocytes, such as antibody secreting B cells or helper T cells and cytolytic T lymphocytes (CTLs). Naïve lymphocytes have surface markers and recirculation patterns that are distinct from those of previously activated lymphocytes.

Memory cells are B or T lymphocytes that mediate rapid and enhanced, i.e. memory (or recall) responses to second and subsequent exposure to antigens. Memory B and T cells are produced by antigen stimulation of naïve lymphocytes and may survive in a functionally quiescent state for many years after the antigen is eliminated.

Dendritic cells are a heterogeneous cell type consisting of multiple subsets. As referred to herein lymph-resident DCs are dendritic cells permanently resident in the lymphoid organs, in particular the CD8+CD205+ subset found in the spleen and lymph nodes of mice. Tissue-derived or trafficking DCs are DCs which are found in non-lymphoid tissue and traffic to lymph nodes upon antigen capture (or spontaneously). Tissue-derived DCs include DCs present in lung and skin.

The invention is described in the examples in relation to influenza virus as the antigen. The inventors propose that the present invention is equally applicable to enhance the immune response to other viral antigens and also to cancer or tumour antigens and antigens from any pathogen, be it of viral, bacterial, fungal or other origin.

An antigen as described herein is any substance that under appropriate conditions results in an immune response in a subject, including, but not limited to, polypeptides, peptides, proteins, glycoproteins, and polysaccharides

Antigens that may be used in the immunogenic composition of the invention include antigens from an animal, a plant, a virus, a protozoan, a parasite, a bacterium, or an antigen associated with a disease state, such as cancer, for example a tumor antigen, or a combination of antigens from the same or different sources.

The immunogenic compositions of the invention may comprise one or more antigens.

The antigen may be any viral peptide, protein, polypeptide, or a fragment thereof derived from a virus including, but not limited to, influenza viral proteins, e.g., influenza virus neuraminidase, influenza virus hemagglutinin, respiratory syncytial virus (RSV)-viral proteins, e.g., RSV F glycoprotein, RSV G glycoprotein, herpes simplex virus (HSV) viral protein, e.g., herpes simplex virus glycoprotein including for example, gB, gC, gD, and gE. Examples of bacterial antigens include the chlamydia MOMP and PorB antigens. Antigen of a pathogenic virus that may be used in the immunogenic compositions of the invention include adenovirdiae (e.g., mastadenovirus and aviadenovirus), herpesviridae (e.g., herpes simplex virus 1, herpes simplex virus 2, herpes simplex virus 5, and herpes simplex virus 6), leviviridae (e.g., levivirus, enterobacteria phase MS2, allolevirus), poxviridae (e.g., chordopoxvirinae, parapoxvirus, avipoxvirus, capripoxvirus, leporiipoxvirus, suipoxvirus, molluscipoxvirus, and entomopoxvirinae), papovaviridae (e.g., polyomavirus and papillomavirus), paramyxoviridae (e.g., paramyxovirus, parainfluenza virus1, mobillivirus (e.g., measles virus), rubulavirus (e.g., mumps virus), pneumonovirinae (e.g., pneumovirus, human respiratory syncytial virus), and metapneumovirus (e.g., avian pneumovirus and human metapneumovirus)), picornaviridae (e.g., enterovirus, rhinovirus, hepatovirus (e.g., human hepatits A virus), cardiovirus, andapthovirus), reoviridae (e.g., orthoreovirus, orbivirus, rotavirus, cypovirus, fijivirus, phytoreovirus, and oryzavirus), retroviridae (e.g., mammalian type B retroviruses, mammalian type C retroviruses, avian type C retroviruses, type D retrovirus group, BLV-HTLV retroviruses, lentivirus (e.g. human immunodeficiency virus 1 and human immunodeficiency virus 2), spumavirus), flaviviridae (e.g., hepatitis C virus), hepadnaviridae (e.g., hepatitis B virus), togaviridae (e.g., alphavirus (e.g., sindbis virus) and rubivirus (e.g., rubella virus)), rhabdoviridae (e.g. vesiculovirus, lyssavirus, ephemerovirus, cytorhabdovirus, and necleorhabdovirus), arenaviridae (e.g., arenavirus, lymphocytic choriomeningitis virus, Ippy virus, and lassa virus), and coronaviridae (e.g., coronavirus andtorovirus).

The antigen may be an infectious disease agent including, but not limited to, influenza virus hemagglutinin, human respiratory syncytial virus G glycoprotein, core protein, matrix protein or other protein of Dengue virus, measles virus hemagglutinin, herpes simplex virus type 2 glycoprotein gB, poliovirus I VP1, envelope glycoproteins of HIV I, hepatitis B surface antigen, diptheria toxin, streptococcus 24M epitope, gonococcal pilin, pseudorabies virus g50 (gpD), pseudorabies virusII (gpB), pseudorabies virusgIII (gpC), pseudorabies virus glycoprotein H, pseudorabies virus glycoprotein E, transmissible gastroenteritis glycoprotein 195, transmissible gastroenteritis matrix protein, swine rotavirus glycoprotein 38, swine parvovirus capsid protein, Serpulinahydodysenteriae protective antigen, bovine viral diarrhea glycoprotein 55, Newcastle disease virus hemagglutinin-neuraminidase, swine flu hemagglutinin, swine flu neuraminidase, foot and mouth disease virus, hog colera virus, swine influenza virus, African swine fever virus, Mycoplasmaliyopneutiioniae, infectious bovine rhinotracheitis virus (e.g., infectious bovine rhinotracheitis virus glycoprotein E or glycoprotein G), or infectious laryngotracheitis virus (e.g., infectious laryngotracheitis virus glycoprotein G or glycoprotein I), a glycoprotein of La Crosse virus, neonatal calf diarrhoea virus, Venezuelan equineencephalomyelitis virus, punta toro virus, murine leukemia virus, mouse mammary tumor virus, hepatitis B virus core protein and/or hepatitis B virus surface antigen or a fragment or derivative thereof, antigen of equine influenza virus or equine herpesvirus (e.g., equine influenza virus type A/Alaska 91 neuraminidase, equine influenza virus typeA/Miami 63 neuraminidase, equine influenza virus type A/Kentucky 81 neuraminidase equine herpesvirus type 1 glycoprotein B, and equine herpesvirus type 1 glycoprotein D, antigen of bovine respiratory syncytial virus or bovine parainfluenza virus (e.g., bovine respiratory syncytial virus attachment protein (BRSV G), bovine respiratory syncytial virus fusion protein (BRSV F), bovine respiratory syncytial virus nucleocapsid protein (BRSVN), bovine parainfluenza virus type 3 fusion protein, and the bovine parainfluenza virus type 3 hemagglutinin neuraminidase), bovine viral diarrhea virus glycoprotein48 or glycoprotein 53.

The antigen may also be a cancer antigen or a tumor antigen. Any cancer or tumor antigen known to one skilled in the art may be used in the present invention including, but not limited to, KS 1/4 pan-carcinoma antigen, ovarian carcinoma antigen (CA125), prostatic acid phosphate, prostate specific antigen, melanoma-associated antigen p97, melanoma antigen gp75, high molecular weight melanoma antigen (HMW-MAA), prostate specific membrane antigen, carcinoembryonic antigen (CEA), polymorphic epithelial mucin antigen, human milk fat globule antigen, colorectal tumor-associated antigens such as: CEA, TAG-72, LEA, Burkitt's lymphoma antigen-38.13, CDl9, human B-lymphoma antigen-CD20, CD33, melanoma specific antigens such as ganglioside GD2, ganglioside GD3, ganglioside GM2, ganglioside GM3, tumor-specific transplantation type of cell-surface antigen (TSTA) such as virally-induced tumor antigens including T-antigen DNA tumor viruses and Envelope antigens of RNA tumor viruses, oncofetal antigen-alpha-fetoprotein such as CEA of colon, bladder tumor oncofetal antigen, differentiation antigen such as human lung carcinoma antigen L6, L20, antigens of fibrosarcoma, human leukemia T cell antigen-Gp37, neoglycoprotein, sphingolipids, breast cancer antigen such as EGFR (Epidermal growth factor receptor), HER2 antigen (pl85HER2), polymorphic epithelial mucin (PEM), malignant human lymphocyte antigen-APO-1, differentiation antigen, such as I antigen found in fetal erythrocytes, primary endoderm, I antigen found in adult erythrocytes, preimplantation embryos, I (Ma) found in gastricadenocarcinomas, M18, M39 found in breast epithelium, SSEA-1 found in myeloid cells, VEP8, VEP9, Myl, VIM-D5, Du56-22 found in colorectal cancer, TRA-1-85 (blood group H), C14 found in colonic adenocarcinoma, F3 found in lung adenocarcinoma, AH6 found in gastric cancer, Y hapten, LeY found in embryonal carcinoma cells, TL5 (blood group A), EGF receptor found in A431 cells, El series (blood group B) found in pancreatic cancer, FC10. 2 found in embryonal carcinoma cells, gastric adenocarcinoma antigen, CO-514 (blood group Lea) found in Adenocarcinoma, NS-10 found in adenocarcinomas, CO-43 (blood groupLeb), G49 found in EGF receptor of A431 cells, MH2 (blood groupALeb/Ley) found in colonic adenocarcinoma, 19.9 found in colon cancer, gastric cancer mucins, TsA7 found in myeloid cells, R24 found in melanoma, 4.2, GD3, Dl.1, OFA-1, GM2, OFA-2, GD2, and M1:22:25:8 found in embryonal carcinoma cells, and SSEA-3 and SSEA-4 found in 4 to 8-cell stage embryos.

The antigen may comprise a virus, against which an immune response is desired. The virus may be a recombinant or chimeric viruses. The virus may be attenuated. Production of recombinant, chimeric and attenuated viruses may be performed using standard methods known to one skilled in the art. The invention encompasses a live recombinant viral antigens or inactivated recombinant viral antigens.

Preferred recombinant viruses are those that are non-pathogenic to the subject to which it is administered. In this regard, the use of genetically engineered viruses for vaccine purposes may require the presence of attenuation characteristics in these strains.

The introduction of appropriate mutations (e.g., deletions) into the templates used for transfection may provide the novel viruses with attenuation characteristics. For example, specific mis-sense mutations which are associated with temperature sensitivity or cold adaption can be made into deletion mutations. These mutations should be more stable than the point mutations associated with cold or temperature sensitive mutants and reversion frequencies should be extremely low.

Alternatively, chimeric viruses with “suicide” characteristics may be constructed for use in the immunogenic compositions of the invention. Such viruses would go through only one or a few rounds of replication within the host. When used as a vaccine, the recombinant virus would go through limited replication cycle(s) and induce a sufficient level of immune response but it would not go further in the human host and cause disease.

Alternatively, inactivated (killed) virus may be used as antigen. Inactivated vaccine formulations may be prepared using conventional techniques to “kill” the chimeric viruses. Inactivated vaccines are “dead” in the sense that their infectivity has been destroyed. Ideally, the infectivity of the virus is destroyed without affecting its immunogenicity. In order to prepare inactivated vaccines, the chimeric virus may be grown in cell culture or in the allantois of the chick embryo, purified by zonal ultracentrifugation, inactivated by formaldehyde or -propiolactone, and pooled.

In certain embodiments, completely foreign epitopes, including antigens derived from other viral or non-viral pathogens can be engineered for use in the immunogenic compositions of the invention. For example, antigens of non-related viruses such as HIV (gpl60, gpl20, gp41) parasite antigens (e.g., malaria), bacterial or fungal antigens or tumor antigens can be engineered into an attenuated strain.

The antigen may include one or more of the select agents and toxins as identified by the Centre for Disease Control. In a specific embodiment, the immunogenic composition may comprise one or more antigens from Staphyloccocal enterotoxin B, Botulinum toxin, protective antigen for Anthrax, and Yersinia pestis. Further antigens will be known to persons skilled in the art.

The immunogenic composition of the present invention may comprise antigens from a single strain, or from a plurality of strains. For example, if the antigen is an influenza virus antigen the immunogenic composition may contain antigens taken from up to three or more viral strains. Purely by way of example an influenza vaccine formulation may contain antigens from one or more strains of influenza A together with antigens from one or more strains of influenza B. Examples of influenza strains are strains of influenza A/Texas/36/91, A/Nanchang/933/95 and B/HarbinZ7/94).

In a most preferred embodiment, the immunogenic composition comprises an influenza virus antigen. In one embodiment the influenza virus antigen is recombinant influenza WSN-gB (H1N1) which contains the gB⁴⁹⁸⁻⁵⁰⁵ K^{b}-restricted epitope of HSV inserted into the neurominidase stalk (Blaney et al., 1998 J. Virol. 12: 9567-74). Other suitable antigens include commercially available influenza vaccine, FLUZONE™, which is an attenuated flu vaccine (Connaught Laboratories, Swiftwater, Pa.). FLUZONE is a trivalent subvirion vaccine comprising 15 μg/dose of each the HAs from influenza A/Texas/36/91 (NINI), A/Beijing/32/92 (H3N2) and B/Panama, 45/90 viruses. The antigen used may be the gB₄₉₈₋₅₀₅ K^{b}-restricted epitope of HSV (SSIEFARL). Persons skilled in the art would recognise suitable influenza virus antigens for use in the present invention.

In accordance with the first aspect of the invention and as referred to herein, a targeting moiety specific for lymph-resident dendritic cells is a moiety that is capable of directing the antigen with which it is associated to lymph-resident DCs in preference to tissue-derived DCs.

Use of the term “specific” is not intended to mean that the targeting moiety is only capable of targeting to lymph-resident DCs, but that it targets lymph-resident DCs in preference to any other DCs, i.e. they are selective for lymph-resident DCS. Such moieties will be known to persons skilled in the art.

The targeting moiety may have a 2×, 4×, 5×, 8×, 10×, 15×, 20×, 30×, 50× or more preference for lymph-resident resident dendritic cells as compared to tissue-derived DCs.

The lymph-resident DC population can be targeted by virtue of the differential expression of surface molecules on DCs. Antibodies raised to these molecules could be used to carry antigen to the lymph-resident DC subset. The primary subset of lymph-resident DC that are to be targeted are CD8+ lymph-resident DC, and more particularly the CD11c+CD8+CD205+CD11b− subset. These can be targeted by the use of antibody-antigen conjugates where the antibody is targeted to CD8alpha or other surface antigens expressed preferentially by this subset. Suitable targeting moieties may include Sca-1 (Spangrude et al, J. Immunol. 141:3697-707, 1988), Sca-2 (Spangrude et al, J. Immunol. 141:3697-707, 1988), CD1d1 (Renukaradhya et al., J. Immunol. 175; 4301-8, 2005), CD36 (Belz et al., J. Immunol. 168: 6066-70, 2002), CD52, CD8alpha, Gpr105 (Moore et al., Brain Res Mol Brain Res 118: 10-23, 2003), and members of G-protein coupled receptor superfamily, Micl (Marshall et al., J Biol Chem 279: 14792-802, 2004) and other C-type lectins and C-type lectin-like molecules, Igsf4 (Galibert et al., J Biol Chem 280: 21955-64, 2005), Treml4 and other members of Ig superfamily and Ig domain containing molecules. Other suitable targeting moieties include necl2 (Galibert et al., 2005, supra), Pslc1 (Qin H. et al., Immunology, 117:419-30, 2006), synCaM (Furuno et al., J Immunol 174: 6934-42, 2005) and sgIgsf (Furuno et al., J Immunol 174: 6934-42, 2005).

Preferably the targeting moiety binds or otherwise associates with a marker on lymph-resident DCs thereby bringing the antigen into proximity with the lymph-resident DCs for antigen processing.

Another way that lymph-resident DCs could be targeted is to use a targeting moiety specific for lymph cells in preference to any other cell or tissue type. Such an approach would also have the effect of bringing the antigen into proximity with lymph-resident DCs for antigen processing.

In accordance with the sixth aspect of the invention and as referred to herein, a targeting moiety specific for tissue-derived dendritic cells is a moiety that is capable of directing the antigen with which it is associated to tissue-derived DCs in preference to lymph-resident DCs. Use of the term specific is not intended to mean that the targeting moiety is only capable of targeting to tissue-derived DCs, but that it targets tissue-derived DCS in preference to any other DCs. Such moieties will be known to persons skilled in the art. The tissue-derived DC population can be targeted by virtue of the differential expression of surface molecules on DCs. Antibodies raised to these molecules could be used to carry antigen to the tissue-derived DC subset. The primary subset of tissue-derived DC that are to be targeted are the CD8−CD205+CD11b− subset or the lung or CD8−CD205+CD11b+subsets of the skin (otherwise known as dermal dendritic cells and Langerhans cells). These can be targeted by the use of antibody-antigen conjugates where the antibody is targeted to tissue-derived DC specific markers such as langerin (Valladeau et al., Immunity 12: 7181, 2000), CD11b (Kurzinger K et al., J. Biol. Chem 257: 12412-8, 1982), Flrt3 (Lacy S E, et al. Genomics. 1999 62:417-26), Interferon induced transmembrane protein 1 (Ishii K, et al. Immunol Lett. 2005 98:280-90.) G protein-coupled receptor 68 (Radu C G, et al. Proc Natl Acad Sci USA. 2005 102:1632-7) Chemokine (C-X-C motif) receptor 4 (Okutsu M, et al. Am J Physiol Regul Integr Comp Physiol. 2005, 288:R591-9)

The targeting moiety may have a 2×, 4×, 5×, 8×, 10×, 15×, 20×, 30×, 50× or more preference for tissue-derived dendritic cells as compared to lymph-resident DCs.

The targeting moiety may be associated with the antigen or bound to the antigen.

As will be recognised by those skilled in the field of protein chemistry there are numerous methods by which the antigen may be bound to the targeting moiety.

### Examples of Such Methods Include:

1) affinity conjugation such as antigen-ligand fusions where the ligand has an affinity for the targeting antibody (examples of such ligands would be streptococcal protein G, staphylococcal protein A, peptostreptococcal protein L) or specific antibody to cross-link antigen to targeting moiety.

2) chemical cross-linking. There are a host of well known cross-linking methods including periodate-borohydride, carbodiimide, glutaraldehyde, photoaffinity labelling, oxirane and various succinimide esters such as maleimidobenzoyl-succinimide ester. Many of these are readily available commercially e.g. from Pierce, Rockford, Ill., USA. There are many references to cross-linking techniques including Hermanson GT “Bioconjugate Techniques” Academic Press, San Diego 1996; Lee Y C, Lee R T. Conjugation of glycopeptides to proteins. Methods Enzymol. 1989; 179: 253-7; Wong S S “Chemistry of Protein Conjugation and Cross-linking” CRC Press 1991; Harlow E & Lane D “Antibodies: A Laboratory Manual” Cold Spring Harbor Laboratory, 1988; Marriott G, Ottl J. Synthesis and applications of heterobifunctional photocleavable cross-linking reagents. Methods Enzymol. 1998; 291: 155-75.

3) genetic fusions. These can be made as recombinant antibody-antigen fusion proteins (in bacteria, yeast, insect or mammalian systems) or used for DNA immunization with or without a linker between the antibody and antigen. There are many publications of immunoglobulin fusions to other molecules. Fusions to antigens like influenza hamagglutinin are known in the art see, for example, Deliyannis G, Boyle J S, Brady J L, Brown L E, Lew A M. “A fusion DNA vaccine that targets antigen-presenting cells increases protection from viral challenge.” Proc Natl. Acad. Sci. USA. 2000 97: 6676-80. Short sequences can also be inserted into the immunoglobulin molecule itself [Lunde E, Western K H, Rasmussen I B, Sandlie I, Bogen B. “Efficient delivery of T cell epitopes to APC by use of MHC class 11-specific Troybodies.” J. Immunol. 2002 168:2154-62]. Shortened versions of antibody molecules (e.g. Fv-fragments) may also be used to make genetic fusions [Reiter Y, Pastan I. “Antibody engineering of recombinant Fv immunotoxins for improved targeting of cancer: disulfide-stabilized Fv immunotoxins.” Clin. Cancer Res. 1996 2: 245-52].

The targeting moiety and antigen may be bonded directly or joined by a linker into a construct. The linker may be a synthetic linker. The linker may be a covalent linkage. The antigen and targeting moiety (and optional linker) may be provided as a fusion protein.

In another embodiment the immunogenic composition may be provided as a nucleic acid construct.

In another embodiment the targeting moiety and antigen are provided separately but associate to allow targeting of the antigen to the appropriate DCs.

The immunogenic composition according to the first aspect of the invention may be used in a booster vaccine and may be provided in a kit optionally together with the primary vaccine. The primary vaccine and booster vaccine may comprise different antigens, although use of the same antigen is preferred.

The vaccine may be a live, attenuated vaccine, an inactivated or “killed” vaccine, a subunit vaccine, a toxoid vaccine, a conjugate vaccine, a DNA-vaccine or a recombinant vector vaccine. Persons skilled in the art of vaccine development will readily understand what is meant by each of these terms.

In some preferred embodiments, the vaccines of the invention are prepared for administration to mammalian subjects in the form of for example, liquids, powders, aerosols, tablets, capsules, enteric coated tablets or capsules, or suppositories. Routes of administration include, without limitation, parenteral administration, intraperitoneal administration, intravenous administration, intramuscular administration, subcutaneous administration, intradermal administration, oral administration, topical administration, intranasal administration, intra-pulmonary administration, rectal administration, vaginal administration, and the like. All such routes are suitable for administration of these compositions, and may be selected depending on the patient and condition treated, and similar factors by an attending physician.

An effective dose of vaccine to be employed therapeutically will depend, for example, upon the therapeutic objectives, the route of administration, and the condition of the subject. Accordingly, it will be necessary for the therapist to titrate the dosage and modify the route of administration as required to obtain the optimal therapeutic effect. A typical daily dosage might range from about 1 mcg/kg to up to 1 mg/kg or more, depending on the mode of delivery.

Dosage levels for the vaccine will usually be of the order of about 50 mcg/kg to about 5 mg per kilogram body weight, with a preferred dosage range between about 0.1 mg to about 1 mg per kilogram body weight per day (from about 0.5 g to about 3 g per patient per day). The amount of active ingredient which may be combined with the carrier materials to produce a single dosage will vary, depending upon the host to be treated and the particular mode of administration. Dosage unit forms will generally contain between from about 5 mg to 500 mg of active ingredient.

It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy.

Selection and upward or downward adjustment of the effective dose is within the skill of the art. The present invention in a first aspect allows for targeting the booster vaccination and accordingly it is expected that a lower dose of booster vaccination will be required to achieve the same level of immune response as obtained with a non-targeted antigen.

Any booster vaccine is desirably administered to the patient about 4 weeks to about 32 weeks following the administration of the priming vaccine. The booster vaccine may be administered via the same route and at the same dosages as provided for the priming vaccine step or at different dosages or via different routes.

The vaccines are desirably formulated into pharmaceutical formulation. Such formulations comprise the antigen and/or targeting moiety combined with a pharmaceutically acceptable carrier, such as sterile water or sterile isotonic saline. The appropriate carrier will be evident to those skilled in the art and will depend in large part upon the route of administration. Formulations include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition. Other parentally-administrable formulations which are useful include those which comprise the active ingredient in microcrystalline form, in a liposomal preparation, or as a component of a biodegradable polymer systems. Formulations for sustained release or implantation may comprise pharmaceutically acceptable polymeric or hydrophobic materials such as an emulsion, an ion exchange resin, a sparingly soluble polymer, or a sparingly soluble salt.

Still additional components that may be present in the formulation are adjuvants, preservatives, chemical stabilizers, or other antigenic proteins. Typically, stabilizers, adjuvants, and preservatives are optimized to determine the best formulation for efficacy in the target human or animal. Suitable exemplary preservatives include chlorobutanol potassium sorbate, sorbic acid, sulfur dioxide, propyl gallate, the parabens, ethyl vanillin, glycerin, phenol, and parachlorophenol. Suitable stabilizing ingredients which may be used include, for example, casamino acids, sucrose, gelatin, phenol red, N-Z amine, monopotassium diphosphate, lactose, lactalbumin hydrolysate, and dried milk. A conventional adjuvant is used to attract leukocytes or enhance an immune response. Such adjuvants include, among others, MPL™ (3-O-deacylated monophosphoryl lipid A; RIBI ImmunoChem Research, Inc., Hamilton, Mont.), mineral oil and water, aluminum hydroxide, Amphigen, Avridine, L121/squalene, D-lactide-polylactide/glycoside, pluronic plyois, muramyl dipeptide, killed Bordetella, saponins, such as Quil A or Stimulon™ QS-21 (Aquila Biopharmaceuticals, Inc., Framingham, Mass.) and cholera toxin (either in a wild-type or mutant form, e.g., wherein the glutamic acid at amino acid position 29 is replaced by another amino acid, preferably a histidine, in accordance with International Patent Application No. PCT/US99/22520).

In one embodiment, the pharmaceutical formulation, if injected has little or no adverse or undesired reaction at the site of the injection, e.g., skin irritation, swelling, rash, necrosis, skin sensitization.

Furthermore, the present invention contemplates a method of making a pharmaceutical formulation comprising admixing the immunogenic composition of the present invention with a pharmaceutically acceptable excipient, vehicle or carrier, and optionally other ingredients.

Also included in the invention is a kit for inducing an enhanced booster immune response. Such a kit preferably comprises the components of a priming vaccine, and the components of the boosting vaccine.

Other components of the kit include applicators for administering each composition. By the term “applicator” as the term is used herein, is meant any device including but not limited to a hypodermic syringe, gene gun, nebulizer, dropper, bronchoscope, suppository, among many well-known types for administration of pharmaceutical compositions useful for administering the DNA vaccine components and/or the protein vaccine components by any suitable route to the human or veterinary patient. Still another component involves instructions for using the kit.

The immunogenic composition of the present invention may be administered to an individual to induce an immune response. Preferably the immune response is enhanced (i.e. is greater) relative to that achieved by administration of antigen alone (without the targeting moiety).

The level of immune response, and thus the potency of the immunogenic composition or vaccine, may be determined by methods known to persons skilled in the art. These could include measurement of CTL responses, antibody responses or helper T cell responses. CTL responses could be measured by the in vivo killer T cell assay (Coles R M, et al. J. Immunol. 2002; 168:834-8), tetramer staining for specific CTL (Altman J. D. et al., Science. 1996; 274:94-6), in vitro restimulation and 51-Cr release assay (Bennett, S. R. et al., J. Exp. Med. 1997; 186:65-70), ELISpots (Yamamoto M., et al., J. Immunol. 1993; 150:106-14) or by Intracellular cytokine staining (Smith et al., Nat. Immunol. 2004; 5:1143-8). Antibody responses could be measured by ELISpot (Czerkinsky C C, et al., J Immunol Methods. 1983; 65:109-21), or ELISA (Engvall E and Perlmann P. J. Immunol. 1972; 109:129-35). Helper T cells responses could be measured by ELISpots (Taguchi T. et al., J Immunol Methods. 1990; 128:65-73), intracellular cytokine staining (Andersson U. and Matsuda T. Eur J. Immunol. 1989 June; 19(6):1157-60) or ELISA for cytokines (Mosmann T. J Immunol Methods. 1983; 65:55-63).

The term “individual” as used herein refers to humans and non-human primates (e.g. gorilla, macaque, marmoset), livestock animals (e.g. sheep, cow, horse, donkey, pig), companion animals (e.g. dog, cat), laboratory test animals (e.g. mouse, rabbit, rat, guinea pig, hamster), captive wild animals (e.g. fox, deer) and any other organisms who can benefit from the immunogenic composition of the present invention. There is no limitation on the type of animal that could benefit from the presently described immunogenic compositions. The most preferred subjects of the present invention are livestock animals and humans. An individual regardless of whether it is a human or non-human may be referred to as a patient, subject, individual, animal, host or recipient.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

It is to be understood that unless otherwise indicated, the subject invention is not limited to specific therapeutic components, manufacturing methods, dosage regimens, or the like, as such may vary. It is also to be understood that the terminology used herein is for the purpose of describing particular embodiments only and is not intended to be limiting.

It must also be noted that, as used in the subject specification, the singular forms “a”, “an” and “the” include plural aspects unless the context clearly dictates otherwise.

### EXAMPLES

The invention is further described in detail by reference to the following experimental examples. These examples are provided for purposes of illustration only, and are not intended to be limiting unless otherwise specified. Thus, the invention encompasses any and all variations which become evident as a result of the teaching provided herein.

### Example 1

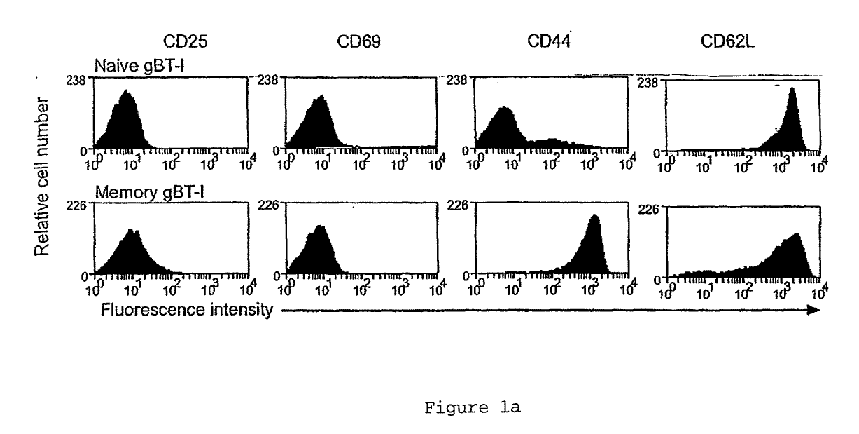

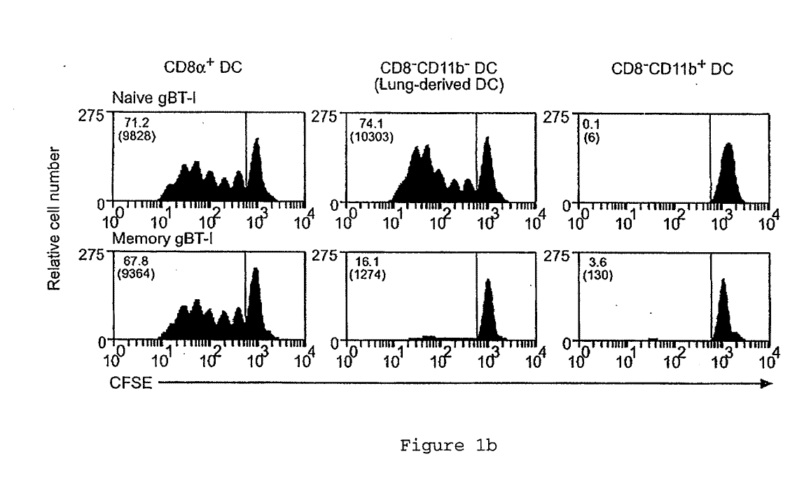

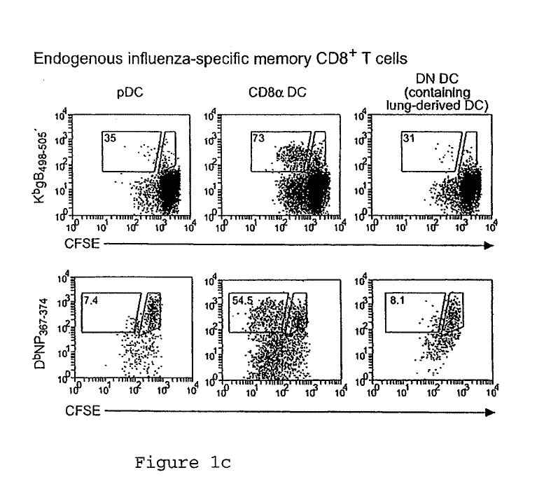

Because memory T cells have been reported to have less co-stimulatory requirements than naïve T cells (Croft, M. et al., J. Immunol. 1994; 152:2675-85), (Byrne, J. A. et al., J. Immunol. 1988; 141:3249-57) we questioned whether memory T cells might respond to additional DC subsets to the two types recognised by naïve T cells during lung infection with influenza virus. To test this, we generated memory T cells by stimulating naïve TCR transgenic CD8⁺ T cells with antigen in vitro and culturing with IL-15 for at least 14 d (FIG. 1a).

We then used these cells as responders to DC isolated ex vivo from the lung-draining lymph nodes of virus infected mice. In these experiments recombinant WSN influenza virus expressing a MHC I-restricted epitope of herpes simplex virus glycoprotein B (gB) was used as the infective agent, termed WSN-gB, and T cells from the gB-specific TCR transgenic mouse, gBT-I, were used as responding CTL. Three days after intranasal infection, at the peak of antigen presentation, CD11⁺c DC were isolated from the mediastinal lymph node, depleted of various cells including plasmcytoid DC (pDC), and separated by CD11b and CD8α expression. Lung-derived DC are CD11b⁻CD8⁻ (CD11b⁻DC), whereas those lymph-resident DC responsible for presenting viral antigens to naïve T cells are CD11b⁻CD8⁺ (CD8⁺ DC). The remaining CD11b⁺ DC are poorly defined, but most likely represent other types of lymph-resident DC.

Unexpectedly, memory T cells were found to be less broadly responsive than naïve T cells, failing to proliferate to antigen presentation by lung-derived DC, though maintaining reactivity to lymph-resident CD8⁺ DC (FIG. 1b). Notably, this was also the case when we produced authentic memory T cells in vivo from a normal T cell repertoire by exposing hosts at least 6 months earlier to virus infection (FIG. 1b), indicating that this finding represents a consistent property of memory CD8⁺ T cells, independent of the method used for their generation. In this latter experiment, DCs were separated based on the expression of CD45RA and CD8, with lung-derived DCs contained within the double negative group (DN DCs), and CD45RA⁺DCs representing pDCs.

### Methods

### Mice

C57BL/6 (H-2^{b}), B6.5JL-PtprcaPep3b/BoyJ (Ly5.1), gBT-I (H-2b) (Mueller, S. et al., Immunol. Cell Biol. 80:156-63, 2002) mice were obtained from The Walter and Eliza Hall Institute of Medical Research animal facility and they were maintained under specific-pathogen free conditions. Experiments with all mice began when they were between 5 and 10 weeks of age according to the guidelines of the Melbourne Directorate Animal Ethics Committee.

### Virus Infections

Mice were anaesthetized with methoxyfluorane and then infected with a non-lethal challenge of recombinant influenza WSN-gB (H1N1) which contains the gB₄₉₈₋₅₀₅ K^{b}-restricted epitope of HSV (SSIEFARL) inserted into the neurominidase stalk (Blaney, J. E. et al., J. Virol. 72:9567-74, 1998). For intranasal infections mice received 10^{2.6} PFU WSN-gB diluted in 25 μl PBS while for intravenous infections mice received 10^{2.95} WSN-gB diluted in 100 μl PBS.

### DC Isolation, Analysis and Culture

DC purification from spleen or LN, analytical and preparative flow cytometry and DC cultures in vitro were carried out as described previously (Belz, G. T. et al., J. Exp. Med. 196:1099-104, 2002; Belz, G. T. et al., J. Immunol. 172:1996-2000, 2004; Belz, G. T. et al., Proc. Natl. Acad. Sci. U.S.A. 101:8670-5, 2004; Allan, R. et al., Science 301:1925-8, 2003; Smith, C. M. et al., Nature Immunol. 5:1143-8, 2004).

### Preparation of CFSE-Labeled CD8⁺ T Cells

Naïve CD8⁺ gBT-I (H-2 K^{b}-restricted anti-gB₄₉₅₋₅₀₅) transgenic T cells were purified from pooled lymph nodes (inguinal, axillary, brachial, superficial cervical and mesenteric) by depletion of non-CD8⁺ T cells as previously described (Belz, G. T. et al., J. Exp. Med. 196:1099-104, 2002; Belz, G. T. et al., J. Immunol. 172:1996-2000, 2004). The T cell populations were routinely 85-95% CD8⁺Vα2⁺ as determined by flow cytometry. Naïve and memory CD8⁺ T cells were labeled with 5,6-CFSE (Belz, G. T. et al., J. Exp. Med. 196:1099-104, 2002; Belz, G. T. et al., J. Immunol. 172:1996-2000, 2004) or used unlabeled. Proliferation was quantitated after 60 h of culture. gBT-I cells were labelled with CD8-specific mAb and resuspended in 100 μL balanced salt solution (BSS)/3% v/v FCS containing 2×10⁴ blank calibration particles (BD Biosciences Pharmingen). The samples were analysed by flow cytometry on a LSR (Beckton Dickinson), and the total number of live dividing lymphocytes (PI^{neg}CFSE^{low}) was calculated from the number of dividing cells per 5×10³ beads.

### Generation of Memory CD8⁺ T Cell Populations

Memory CD8⁺ T cells were created using an established model for the in vitro differentiation of central memory T cells (Belz, G. T. et al., Eur. J. Immunol. 36:327-35, 2006; Manjunath, N. et al., J. Clin. Invest. 108:871-8, 2001; Klebanoff, Proc. Natl. Acad. Sci. U.S.A. 102:9571-6, 2005; Klebanoff, C. A. Proc. Natl. Acad. Sci. U.S.A. 101:1969-74, 2004; Wong, P. and Palmer E. G. Immunity 18:499-511, 2003). Naïve gBT-I transgenic CD8⁺ T cells were coated with 1 μM gB peptide for 1 hour at 37° C. Cells were then washed twice in hepes earl media containing 2.5% FCS before culture at 1.7×10⁵ cells/ml in complete mouse tonicity RPMI 1640 medium (RPMI-1640 containing 10% FCS, 50 μM 2ME, 2 mM L-glutamine, 100 U/ml penicillin and 100 μg/ml streptomycin, complete medium). After 2 days, cells were washed and supplemented with recombinant hIL15 (20 ng/ml) (R&D Systems, Minneapolis, Minn. 55413 USA). Complete media containing hIL15 was replaced every 3-4 days and cells were used between 14 and 20 days after initiation of the culture.

### Results

FIG. 1 shows that naïve but not memory CD8⁺ T cells proliferate in response to lung-derived (CD8⁻CD11b⁻) DC from the mediastinal LN of influenza virus-infected mice. FIG. 1a shows the phenotype of naïve and memory CD8⁺ T cells. Naïve gBT-I cells isolated directly ex vivo, and cells activated in vitro for 17 days (memory) were analysed for expression of activation markers CD25, CD69, CD44 and CD62L to confirm the developmental phenotype of the cells as activated effector or memory cells. Conventional CD8⁺CD11b⁻ DC, CD8⁻CD11b⁻ DC and CD8⁻CD11b⁺ DC were isolated from mediastinal LN of mice three days after intranasal infection with 400 PFU WSN-gB. Purified DC were co-cultured for 60 h with CD8⁺ CFSE-labelled naïve or memory transgenic CD8⁺ T cells specific for gB before analysis by flow cytometry. The histograms shown in FIG. 1b are representative of 4 experiments with similar results.

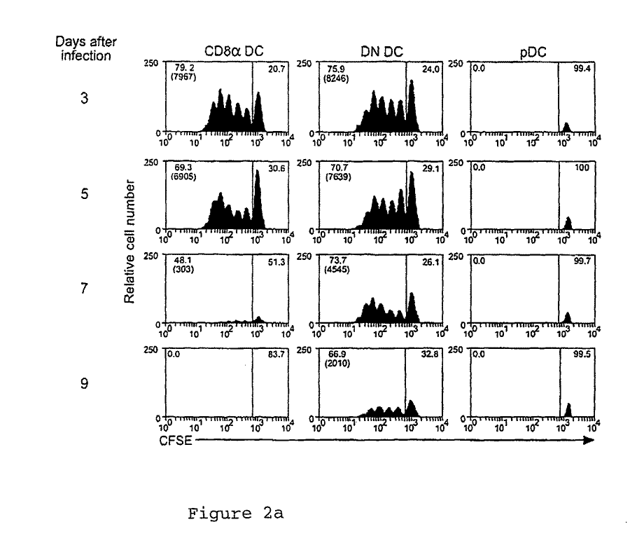

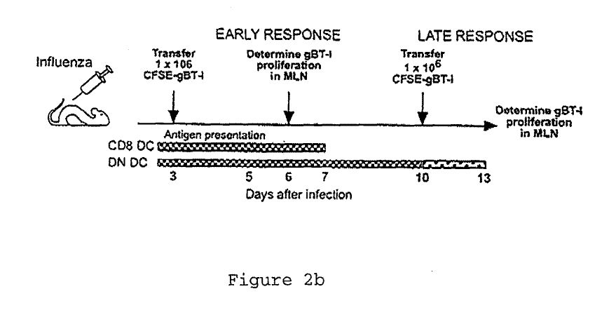

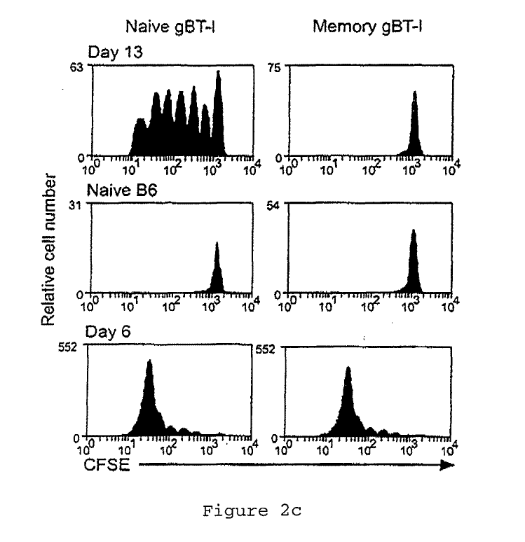

Examination of the time course of ex vivo antigen presentation by DC to naïve T cells after influenza virus infection showed that while lymph-resident CD8 DC only presented viral antigens for the first 7 days, lung-derived DC (again, contained in the DN DC group as used in FIG. 1c) could present antigens for at least 9 days (FIG. 2a). DC were isolated from the MLN, stained for CD8 and CD45RA and sorted into CD45RA+ DC (pDC) or CD45RA− DC that were either CD8⁺ (CD8 DC) or CD8⁻ (DN DC) (20 donor mice per timepoint) at various times after infection. These DC were cultured with naive gBT-I CD8⁺ CFSE-labelled transgenic T cells specific for herpes simplex viral glycoprotein B (gB). After 60 hr, cultures were analysed for T cell proliferation as measured by dilution of CFSE. Analyses of days 3, 5, 7 and 9 after infection were performed within the same experiment. This experiment was performed twice with similar findings, as shown in FIG. 2a. In this experiment, DC were separated by expression of CD45RA and CD8, with lung-derived DC contained within the double negative group (DN DC), and CD45RA⁺ DC representing pDC. If, as suggested from the data above, memory T cells are only capable of responding to lymph-resident CD8⁺ DC, then naïve T cells, but not memory T cells, should respond in vivo at time points later than day 7—when only lung-derived DC would be presenting viral antigens. To test this, mice were infected intranasally with WSN-gB and then 10 days later injected with CFSE-labeled naïve or memory gBT-I cells to examine proliferation in vivo 60 h later (FIGS. 2b, c). As a positive control, CFSE-labeled T cells were also injected on day 3 of infection when lymph-resident DC should be capable of stimulating both naïve and memory T cells (FIGS. 2b, d). Consistent with our notion, both T cell populations responded when injected on day 3 (FIG. 2c, lower panel), but only the naïve T cells proliferated at day 10 (FIG. 2c, upper panel). These data confirmed our ex vivo findings, showing that in vivo the lung-derived DCs failed to stimulate memory T cells, though they were capable of activating naïve T cells. The capacity of memory T cells to respond on day 3 (FIG. 2c, lower panel), and the detection of these cells in the LN after transfer on day 10 (FIG. 2c, middle panel), confirmed that this population of cells were able to home to lymph nodes.

Accordingly prolonged antigen presentation by lung-derived DC allows in vivo expansion of naïve but not memory antigen-specific CD8⁺ T cells late in infection.

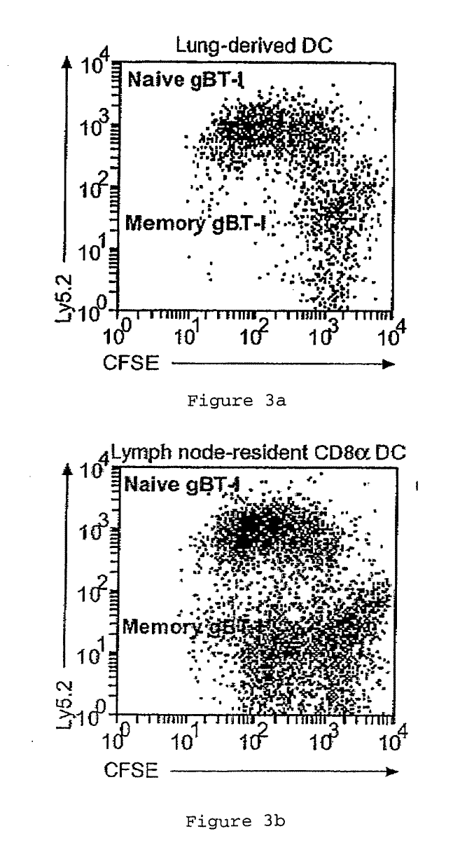

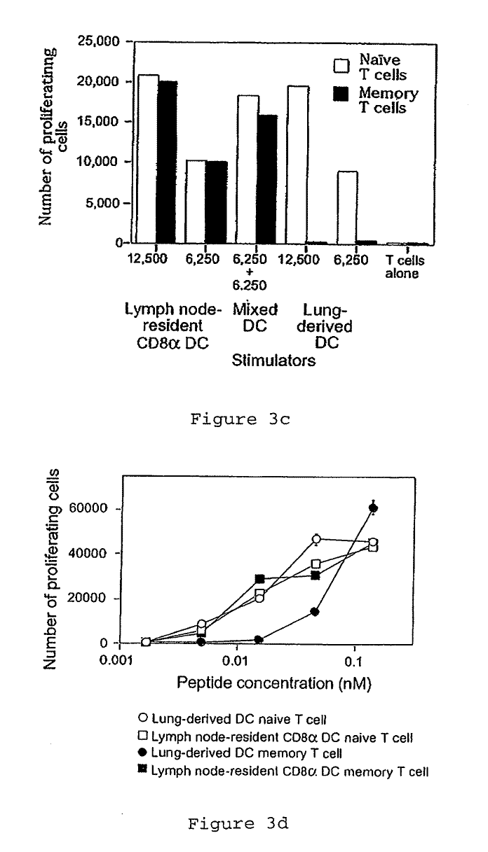

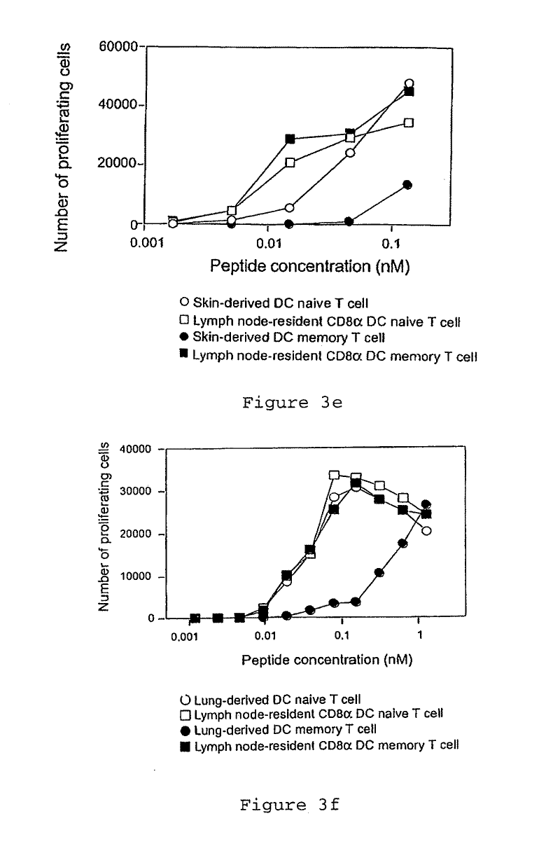

One trivial explanation for the observations was that memory T cells kill lung-derived DC or suppress their function, preventing them from inducing proliferation. This was tested by co-culturing lung-derived DC with a mixture of naïve and memory T cells (FIG. 3a). This showed that while, as expected, memory T cells failed to respond, naïve T cells still proliferated. In contrast, lymph-resident CD8+DC stimulated both naïve and memory T cells when cultured together (FIG. 3b).

Conversely, to determine whether the poor response of memory T cells to lung-derived DCs could be explained by suppressive factors provided by the DCs themselves, we compared the response of memory T cells to lymph node-resident DCs in the presence or absence of lung-derived DCs (FIG. 3c).

The capacity of DC subtypes to stimulate naïve and memory CD8⁺ T cells is shown in FIG. 3. Responsiveness of mixed cultures of naïve and memory T cells to different DC subtypes is shown in FIGS. 3a,b. FIG. 3c shows the responsiveness of naïve and memory T cells to mixtures of CD8 DC and CD11b⁻ DC from MLN. FIG. 3d shows the responsiveness of naïve and memory T cells to peptide coated CD8 DC and CD11b⁻ DC from MLN. FIG. 3e shows the responsiveness of naïve and memory T cells to peptide-coated CD8⁺ and CD8⁻DEC205⁺ (Langerhans cells and dermal) DC from skin draining LN. In FIGS. 3d,e DC (5×10³) were coated for 60 min with titrating concentrations of SSIEFARL peptide, washed three times and then cultured for 60 with 5×10⁴ CFSE-labelled CD8⁺ gBT-I transgenic T cells. Proliferated gBT-I CD8⁺ T cells were counted by flow cytometry. Data are one representative of two experiments for each data set. Lung-derived DCs did not impair responses to lymph node-resident DCs, suggesting an overt suppressive mechanism was not operative.

To more quantitatively assess the difference in stimulatory capacity of lung-derived DC for naïve and memory T cells, we isolated both lymph-resident CD8 DC and lung-derived DC from uninfected mice, coated them with various concentrations of gB peptide and examined their ability to stimulate naïve and memory gBT-I T cells (FIG. 3d). This revealed an equivalent response by both naïve and memory T cells to lymph-resident DC, but an approximately 10-fold reduction in the sensitivity of memory T cells to lung-derived DC. Similar results were evident when DCs from virus-infected mice were used (FIG. 3f). Thus, while lung-derived DC could not stimulate memory T cells to influenza virus during infection, this was not due to a complete failure of this population to activate memory, but rather a 10-fold reduced capacity to stimulate. Realistically, however, this difference could mean that most natural stimuli are ineffective at stimulating memory T cells when presented on lung-derived DC.

These findings extended beyond the lung-derived DC, reproduced by comparing peptide presentation by skin-derived DC (FIG. 3d). Again, while lymph-resident DC stimulated both naïve and memory cells equivalently, skin-derived DC (consisting of a mixture of dermal DC and Langerhans cells) were 10-fold less efficient at stimulating memory T cells.

### Example 2

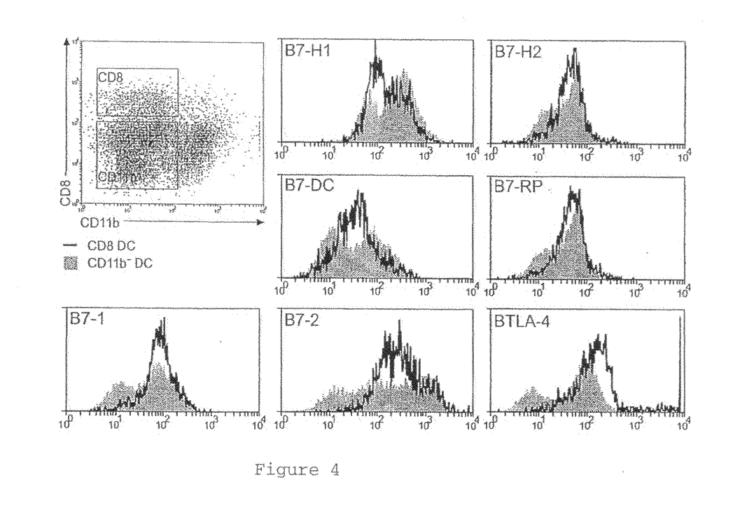

Having established that tissue-derived DC (from lung or skin) could stimulate naïve T cells more efficiently than memory T cells, we asked whether they could prime naïve responses when large numbers of competing memory cells were present. This might be advantageous if pre-existing memory populations derived, for example, from cross-reactive infections were not particularly protective. This was tested by injecting small numbers (5×10⁴) of naïve gBT-I T cells (responding population) and examining their in vivo expansion in response to WSN-gB infection in the presence of titrated numbers of memory gBT-I cells (competitor population) (FIG. 4a-d). Following lung infection with WSB-gB, competition exists between naïve competitor and naïve responder CD8⁺ T cells (FIG. 4a), naïve competitor and memory responder CD8⁺ T cells (FIG. 4b), but not between memory competitor and naïve responder CD8⁺ T cells (FIG. 4c). Competition was observed between memory competitor and naïve responder CD8⁺ T cells following intravenous infection with WSN-gB, where no tissue-derived DC present antigen. In these experiments, titrating numbers of naïve or memory CD8⁺ T cells (competitors) were adoptively transferred into naïve hosts together with 5×10⁴ naïve or memory responder CD8⁺ T cells. Mice were either infected intranasally (FIG. 4 a-c) with WSN-gB and their tissues analysed ten days later, or they were infected intravenously (FIG. 4d) and analysed 8 days later by flow cytometry for the number of gB-specific CD8⁺ responder cells generated during the infection. The data presented are show results of individual experiments with at least two mice per experimental point.

The responding population was identified by an Ly5 allotypic marker and the number of cells generated in response to infection assess on day 8-10. As a control, we first showed that naïve T cells competed well with other naïve T cells (FIG. 4a). As a second control, we showed that naïve T cells competed very well with a responding population of memory T cells, preventing their expansion when in excess (FIG. 4b). This was expected, as memory T cells should only be able to recognise antigen on DC that can also present to naïve T cells, i.e. the lymph-resident DC. Importantly, however, when we compared the ability of increasing numbers of memory T cells to compete with a naïve responding population, memory T cells were unable to compete (FIG. 4c) although their presence was clearly evident.

To support the view that this was because trafficking DC presented viral antigens to naïve but not memory T cells, we examined competition under circumstances where trafficking DC were not involved. Intravenous viral infection results in presentation by the lymph-resident CD8+ DC only, which stimulate memory and naïve T cells equally (FIG. 3). In contrast to lung infection (FIG. 4c), when mice were infected intravenously with WSN-gB (FIG. 4d), increasing numbers of memory T cells were able to prevent naïve responses. Together, these data indicate that trafficking DC are critical for naïve T cell stimulation when competing memory cells are present. This explains why naïve T cell responses could be detected despite the presence of preformed memory for lung infection with influenza virus.

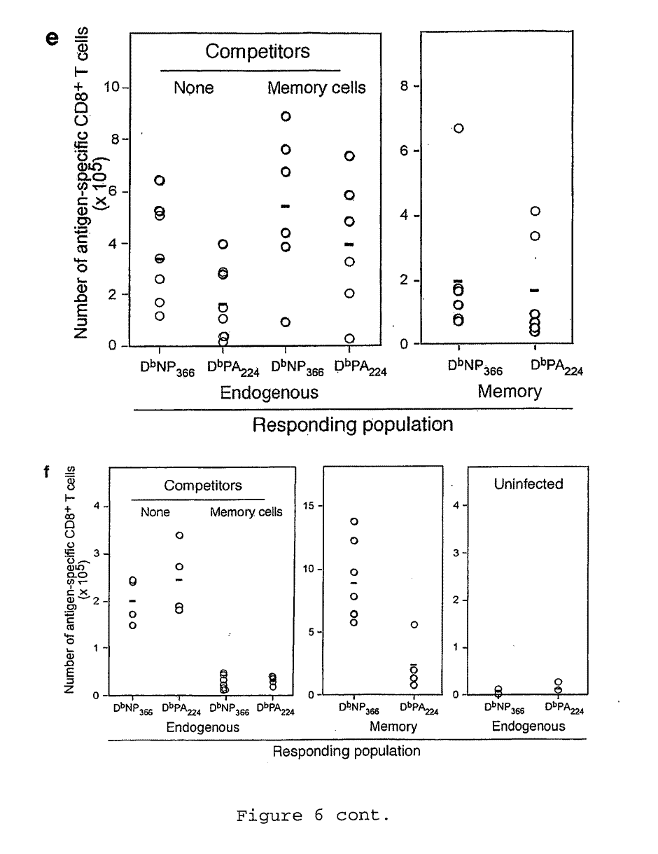

To verify these findings using authentic (rather than transgenic) T cells, we isolated CD44^{hi}CD62L^{hi} central memory T cells from B6.Ly5.1 mice at least 12 wks after infection with HKx31 influenza virus and used these as competitors by adoptive transfer into B6 mice. These mice were subsequently infected intranasally (FIG. 6e) or intravenously (FIG. 6f) with influenza virus and then we examined the response by endogenous and transferred cells specific for viral NP and PA. Consistent with studies using transgenic T cells, this showed that memory CD8 T cells prevented the response of naïve endogenous T cells to influenza virus after intravenous infection, but not after lung infection. Together, these data indicate that tissue-derived DCs provide a preferential avenue for naïve T cell stimulation when competing memory cells are present.

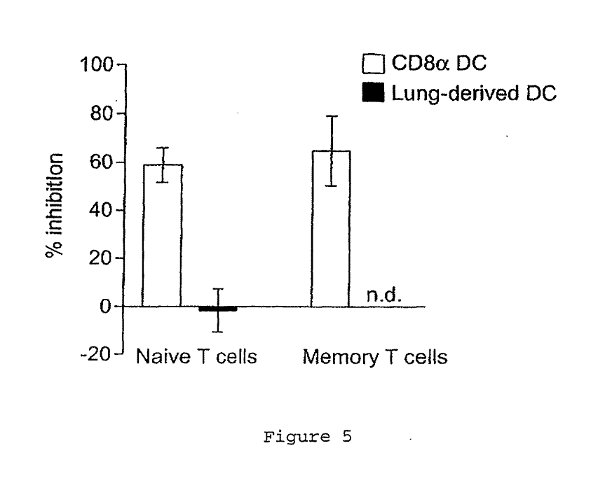

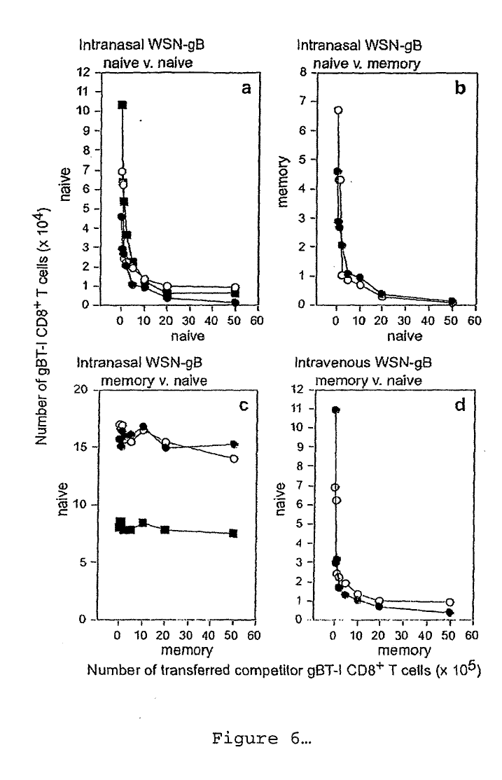

These data provide other important conclusions. Naïve CD8⁺ T cells are shown to be more sensitive than memory CD8⁺ T cells for stimulation by tissue-derived DC, and are equivalent to naïve T cells in their response to lymph-resident DC (FIG. 3). This questions the long-held paradigm that memory T cells have fewer costimulatory requirements than naïve T cells, at least when tissue-derived DCs are used as antigen presenting cells. How this is achieved at the molecular level is unclear, though we have excluded obvious differences in expression of various co-stimulatory molecules including B7-H1, B7-H2, B7-DC, B7-RP, B7-1, B7-2 and BTLA-4 (FIG. 4). Based on DC subset diversity in their use of CD70, we examined the role of this molecule in our study. Interestingly, responses induced by lymphoid-resident CD8a DCs were CD70-dependent for both naïve and memory T cells, while the stimulation of naïve T cells by lung-derived DCs was CD70 independent (FIG. 5). This implied that lung-derived DCs use an alternative as yet undefined costimulatory molecule to efficiently stimulate naive T cells, but that this signal inefficiently stimulates memory T cells.

### Example 3

In vivo and in vitro-derived memory T cells respond similar to endogenous memory T cells (FIG. 7). CD8⁺ DC, CD11b⁻ DC and CDl1b+DC were isolated from mediastinal LN of mice three days after intranasal infection with 400 PFU WSN-gB. Purified DC were co-cultured for 60 h with gBT-I CD8⁺ CFSE-labelled naïve or memory transgenic CD8⁺ T cells specific for gB before analysis by flow cytometry. Memory T cells were generated by three different methods. Briefly, (second row) naïve gBT-I cells were transferred into Rag1−/− mice that were infected intranasally with WSN-gB and 8 10 wk later, their spleen was harvested, and memory gBT-I CD8+ T cells purified. Third row, memory gBT-I cells were prepared as described in Methods above, by peptide antigen stimulation in Vitro and maintenance using recombinant human IL-15. Bottom row, B6 mice were infected intranasally with WSN-gB and, 8-10 wk later, endogenous non-transgenic CD8+ T cells were purified.

The results are provided in FIG. 7. The histograms are representative of 2 experiments with similar results and show proliferation of the T cell population (⅓ well collected). The percent and number (parenthesis) of proliferating cells for each plot are indicated.

Our findings imply that memory T cells are highly dependent on presentation by the lymph-resident DC, since their ability to respond to trafficking DC is compromised. This differential responsiveness may be important when weakly cross-reactive and ineffective memory T cells generated to an earlier virus are available. The mechanism we describe here provides a means to circumvent competition by dominant but ineffective memory T cells, since naïve T cells capable of fighting infection will also have an opportunity to be stimulated. Such cross-reactivity is likely to be rare for two different species of pathogen, but for viruses that are able to mutate T cell epitopes such as influenza virus (Voeten, J. T. et al., J. Virol. 74:6800-07, 2000), HIV (Phillips, R. E. et al., Nature 354:453-9, 1991), and Hepatitis C virus (Weiner, A. et al., Proc. Natl. Acad. Sci. U.S.A. 92:2755-9, 1995), this is likely to be more common, though both instances have been documented.

Our studies highlight differences in the way memory and naïve CD8⁺ T cells interact with DC subsets, providing confronting evidence that naïve T cells may have fewer requirements for activation than memory T cells. These findings not only justify further scrutiny of the precise functions of individual DC subsets, but they provide insight into novel strategies for vaccine development. Clearly, in prime-boost strategies, targeting booster antigen to lymph-resident DC would be beneficial.

1. An immunogenic composition for raising an immune response to an antigen, the composition comprising the antigen and a targeting moiety specific for lymph-resident dendritic cells.

2. An immunogenic composition according to claim 1, in which the antigen is a viral antigen, a cancer or tumour antigen or an antigen from any pathogen of viral, bacterial, fungal or other origin.

3. An immunogenic composition according to claim 1, in which the antigen is a virus, a viral peptide, protein, polypeptide, or a fragment thereof derived from a virus.

4. An immunogenic composition according to claim 3, in which the virus is selected from influenza virus, respiratory syncytial virus (RSV), chlamydia, adenovirdiae, mastadenovirus, aviadenovirus, herpesviridae, herpes simplex virus 1, herpes simplex virus 2, herpes simplex virus 5, herpes simplex virus 6, leviviridae, levivirus, enterobacteria phase MS2, allolevirus, poxviridae, chordopoxvirinae, parapoxvirus, avipoxvirus, capripoxvirus, leporiipoxvirus, suipoxvirus, molluscipoxvirus, entomopoxvirinae, papovaviridae, polyomavirus, papillomavirus, paramyxoviridae, paramyxovirus, parainfluenza virus 1, mobillivirus, measles virus, rubulavirus, mumps virus, pneumonovirinae, pneumovirus, metapneumovirus, avian pneumovirus, human metapneumovirus, picornaviridae, enterovirus, rhinovirus, hepatovirus, human hepatitis A virus, cardiovirus, andapthovirus, reoviridae, orthoreovirus, orbivirus, rotavirus, cypovirus, fijivirus, phytoreovirus, oryzavirus, retroviridae, mammalian type B retroviruses, mammalian type C retroviruses, avian type C retroviruses, type D retrovirus group, BLV-HTLV retroviruses, lentivirus, human immunodeficiency virus 1, human immunodeficiency virus 2, spumavirus, flaviviridae, hepatitis C virus, hepadnaviridae, hepatitis B virus, togaviridae, alphavirus sindbis virus, rubivirus, rubella virus, rhabdoviridae, vesiculovirus, lyssavirus, ephemerovirus, cytorhabdovirus, necleorhabdovirus, arenaviridae, arenavirus, lymphocytic choriomeningitis virus, Ippy virus, lassa virus, coronaviridae, coronavirus and torovirus.

5. An immunogenic composition according to claim 3, in which the viral peptide, protein, polypeptide, or a fragment thereof is selected from influenza virus neuraminidase, influenza virus hemagglutinin, respiratory syncytial virus (RSV)-viral proteins, RSV F glycoprotein, RSV G glycoprotein, herpes simplex virus (HSV) viral proteins, herpes simplex virus glycoproteins gB, gC, gD, and gE, and chlamydia MOMP and PorB antigens.

6. An immunogenic composition according to claim 1, in which the antigen is a infectious disease agent selected from influenza virus hemagglutinin, human respiratory syncytial virus G glycoprotein, core protein, matrix protein or other protein of Dengue virus, measles virus hemagglutinin, herpes simplex virus type 2 glycoprotein gB, poliovirus I VP1, envelope glycoproteins of HIV I, hepatitis B surface antigen, diptheria toxin, streptococcus 24M epitope, gonococcal pilin, pseudorabies virus g50 (gpD), pseudorabies virus II (gpB), pseudorabies virusgIII (gpC), pseudorabies virus glycoprotein H, pseudorabies virus glycoprotein E, transmissible gastroenteritis glycoprotein 195, transmissible gastroenteritis matrix protein, swine rotavirus glycoprotein 38, swine parvovirus capsid protein, Serpulinahydodysenteriae protective antigen, bovine viral diarrhea glycoprotein 55, Newcastle disease virus hemagglutinin-neuraminidase, swine flu hemagglutinin, swine flu neuraminidase, foot and mouth disease virus, hog colera virus, swine influenza virus, African swine fever virus, Mycoplasma liyopneutiioniae, infectious bovine rhinotracheitis virus, infectious bovine rhinotracheitis virus glycoprotein E, glycoprotein G, infectious laryngotracheitis virus, infectious laryngotracheitis virus glycoprotein G or glycoprotein I, a glycoprotein of La Crosse virus, neonatal calf diarrhoea virus, Venezuelan equine encephalomyelitis virus, punta toro virus, murine leukemia virus, mouse mammary tumor virus, hepatitis B virus core protein and hepatitis B virus surface antigen or a fragment or derivative thereof, antigen of equine influenza virus or equine herpes virus, including equine influenza virus type A/Alaska 91 neuraminidase, equine influenza virus typeA/Miami 63 neuraminidase, equine influenza virus type A/Kentucky 81 neuraminidase equine herpes virus type 1 glycoprotein B, and equine herpes virus type 1 glycoprotein D, antigen of bovine respiratory syncytial virus or bovine parainfluenza virus, bovine respiratory syncytial virus attachment protein (BRSV G), bovine respiratory syncytial virus fusion protein (BRSV F), bovine respiratory syncytial virus nucleocapsid protein (BRSVN), bovine parainfluenza virus type 3 fusion protein, bovine parainfluenza virus type 3 hemagglutinin neuraminidase, bovine viral diarrhoea virus glycoprotein 48 and glycoprotein 53.

7. An immunogenic composition according to claim 1, in which the antigen is a cancer antigen or a tumor antigen.

8. An immunogenic composition according to claim 7, in which the cancer or tumor antigen is selected from KS 1/4 pan-carcinoma antigen, ovarian carcinoma antigen (CA125), prostatic acid phosphate, prostate specific antigen, melanoma-associated antigen p97, melanoma antigen gp75, high molecular weight melanoma antigen (HMW-MAA), prostate specific membrane antigen, carcinoembryonic antigen (CEA), polymorphic epithelial mucin antigen, human milk fat globule antigen, colorectal tumor-associated antigens, CEA, TAG-72, LEA, Burkitt's lymphoma antigen-38.13, CD19, human B-lymphoma antigen-CD20, CD33, melanoma specific antigens, ganglioside GD2, ganglioside GD3, ganglioside GM2, ganglioside GM3, tumor-specific transplantation type of cell-surface antigen (TSTA), virally-induced tumor antigens, T-antigen DNA tumor viruses, Envelope antigens of RNA tumor viruses, oncofetal antigen-alpha-fetoprotein, CEA of colon, bladder tumor oncofetal antigen, differentiation antigen, human lung carcinoma antigen L6, L20, antigens of fibrosarcoma, human leukemia T cell antigen-Gp37, neoglycoprotein, sphingolipids, breast cancer antigen, EGFR (Epidermal growth factor receptor), HER2 antigen (pl85HER2), polymorphic epithelial mucin (PEM), malignant human lymphocyte antigen-APO-1, differentiation antigen, including I antigen found in fetal erythrocytes, primary endoderm, I antigen found in adult erythrocytes, preimplantation embryos, I (Ma) found in gastricadenocarcinomas, M18, M39 found in breast epithelium, SSEA-1 found in myeloid cells, VEP8, VEP9, Myl, VIM-D5, Du56-22 found in colorectal cancer, TRA-1-85 (blood group H), C14 found in colonic adenocarcinoma, F3 found in lung adenocarcinoma, AH6 found in gastric cancer, Y hapten, LeY found in embryonal carcinoma cells, TL5 (blood group A), EGF receptor found in A431 cells, El series (blood group B) found in pancreatic cancer, FC10. 2 found in embryonal carcinoma cells, gastric adenocarcinoma antigen, CO-514 (blood group Lea) found in Adenocarcinoma, NS-10 found in adenocarcinomas, CO-43 (blood groupLeb), G49 found in EGF receptor of A431 cells, MH2 (blood groupALeb/Ley) found in colonic adenocarcinoma, 19.9 found in colon cancer, gastric cancer mucins, TsA7 found in myeloid cells, R24 found in melanoma, 4.2, GD3, D1.1, OFA-1, GM2, OFA-2, GD2, and M1:22:25:8 found in embryonal carcinoma cells, and SSEA-3 and SSEA-4 found in 4 to 8-cell stage embryos.

9. An immunogenic composition according to claim 3, comprising a recombinant or chimeric virus.

10. An immunogenic composition according to claim 9, in which the virus comprises a live recombinant viral antigens or inactivated recombinant viral antigens.

11. An immunogenic composition according to claim 1, in which the antigen comprises one or more antigens from Staphyloccocal enterotoxin B, Botulinum toxin, protective antigen for Anthrax, and Yersinia pestis.

12. An immunogenic composition according to claim 3, comprising antigens from a single virus strain, or from a plurality of strains.

13. An immunogenic composition according to claim 1, in which the antigen comprises an influenza virus antigen.

14. An immunogenic composition according to claim 13, in which the antigen is recombinant influenza WSN-gB (H1N1) which contains the gB⁴⁹⁸⁻⁵⁰⁵ K^{b}-restricted epitope of HSV inserted into the neurominidase stalk or comprising HAs from influenza A/Texas/36/91 (NINI), A/Beijing/32/92 (H3N2) and B/Panama, 45/90 viruses.

15. An immunogenic composition according to claim 1, in which the antigen is the gB₄₉₈₋₅₀₅ K^{b}-restricted epitope of HSV (SSIEFARL).

16. An immunogenic composition according to claim 1 in which the target moiety is selected from Sca-1, Sca-2, CD1d1, CD36, CD52, CD8α, Gpr105, and members of G-protein coupled receptor superfamily, Micl and other C-type lectins and C-type lectin-like molecules, Igsf4, Treml4 and other members of Ig superfamily and Ig domain containing molecules, nec12, Pslc1, synCaM and sgIgsf.

17. A booster vaccine comprising an immunogenic composition according to claim 1.

18. A kit comprising a first vaccine and a booster vaccine comprising the immunogenic composition according to claim 1.

19. A method of inducing an immune response in an individual comprising administering to the individual a booster vaccine according to claim 17.

20. A method according to claim 19, in which the immune response comprises a killer T cell response.

21. (canceled)

22. (canceled)

23. (canceled)

24. (canceled)
